(19) United States
(12) Patent Application Publication
(10) Pub. No.: US 2005/0181475 A1
(43) Pub. Date: Aug. 18, 2005

(54) Immunogenic monoclonal antibody

(75) Inventors: Hans Loibner, Wien (AT); Gunter Waxenecker, Mank (AT); Gottfried Himmler, Wien (AT); Helmut Eckert, Obervil (CH); Manfred Schuster, Schrick (AT); Ralf Kircheis, Wien (AT)

Correspondence Address: BIRCH STEWART KOLASCH & BIRCH, PO BOX 747, FALLS CHURCH, VA, 22040-0747, US

(73) Assignee: Igeneon Krebs-Immuntherapie Forschungs-Und-Entwicklungs-AG, Vienna (AT)

(21) Appl. No.: 10/514,529

(22) PCT Filed: May 15, 2003
(86) PCT No.: PCT/AT03/00142

(30) Foreign Application Priority Data
May 15, 2002 (AT) A 744/2002

(51) Int. Cl. C12P 21/06; C07H 21/04; A61K 39/395; C07K 16/30; C12N 5/06

(52) U.S. Cl. 435/69.1; 435/320.1; 435/338; 530/388.8; 424/155.1; 536/23.53

(57) ABSTRACT

The invention relates to an immunogenic antibody which comprises at least two different epitopes of a tumor-associated antigen.

The present invention relates to monoclonal antibodies suitable for the preparation of tumor vaccines as well as a method for immunogenizing tumor-associated antigens.

In addition to other physiological peculiarities that distinguish cancer cells from normal cells, cancer cells virtually always have a modified type of glycosylation (Glycoconj. J. (1997), 14:569; Adv. Cancer Res. (1989), 52:257; Cancer Res. (1996), 56:5309). Although modifications differ from one tissue to another, it has been observed that a modified glycosylation is typical of cancer cells. In most cases, the modified glycosylation is presented on the surface of the cells in the form of glycoproteins and glycolipids. These modified sugar structures may, therefore, be referred to as tumor-associated antigens (TAAs), which in many cases are sufficiently tumor-specific, i.e. occur rarely in “normal” cells. In many cases cells, and also tumor cells, do not produce uniform glycosylation, i.e., there are various glycoforms of complex glycan chains on one cell (Annu. Rev. Biochem. (1988), 57:785).

Examples of tumor-associated carbohydrate structures are Lewis antigens, which are expressed to an increasing extent in many epithelial types of cancer. They include Lewis X, Lewis B and Lewis Y structures as well as sialylated Lewis X structures. Other carbohydrate antigens are GloboH structures, KH1, Tn antigen, TF antigen, alpha-1,3-galactosyl epitope (Elektrophoresis (1999), 20:362; Curr. Pharmaceutical Design (2000), 6:485, Neoplasma (1996), 43:285).

Other TAAs are comprises of proteins that are especially strongly expressed on cancer cells, such as, e.g., CEA, TAG-72, MUC1, folate binding protein A-33, CA125, EpCAM, HER-2/neu, PSA, MART, etc. (Sem. Cancer Biol. (1995), 6:321).

One approach to destroying tumor cells in a relatively specific manner is a passive immunotherapy with antibodies directed against TAAs (Immunology Today (2000), 21:403-410; Curr. Opin. Immunol. (1997), 9:717).

Another approach to destroying tumor cells is an active vaccination that will trigger an immune response to TAA. Such an immune response will, thus, also be directed against the respective tumor cells (Ann. Med. (1999), 31:66; Immunobiol. (1999), 201:1).

In the event of an active immunization, the weak immunogenity of antigens constitutes a big problem. Carbohydrates are very small molecules and, therefore, will not be directly recognized by the immune system. Carbohydrates and polysaccharides, in general, are regarded as thymus-independent antigens. The conjugation of immunologically inert carbohydrate structures with thymus-dependent antigens such as proteins, will enhance their immunogenity. Vaccines based on tumor-associated carbohydrate structures are, therefore, coupled to so-called “carrier molecules” in order to enhance their immunogenity (Angw. Chem. Int. Ed. (2000), 39:836). Proteins like bovine serum albumin or KLH (keyhole limpet hemocyanin) often serve as carrier molecules. The protein will stimulate the carrier-specific T-helper cells, which will then play a role in the induction of the anti-carbohydrate-antibody synthesis (Contrib. Microbiol. Immunol. (1989), 10:18).

Moreover, both carbohydrate antigens and protein antigens are also present on healthy tissues (at least in particular stages of development of the organism) and will, therefore, be recognized as autologous material by the immune system—consequently, no immune response will as a rule be available against those endogenous molecules.

Another option to improve the quality of an immune response to carbohydrates is the immunization with so-called “mimotopes” which are no carbohydrates (e.g., peptides; Nat. Biotechnol. (1999), 17:660; Nat. Biotechnol. (1997), 15:512).

Tumor-associated proteins are hardly immunogenic, but are nevertheless taken into consideration as vaccines (Ann. Med. (1999), 31:66; Cancer Immunol. Immunother. (2000), 49:123; U.S. Pat. No. 5,994,523). A way of avoiding the “self”-recognition of specific TAAs resides in the use of anti-idiotypical antibodies as immunogens, which imitate the structure of a TAA, thus triggering an immune response that will also react with the TAA (Cancer Immunol. Immunother. (2000), 49:133). There are still other ways of breaking the self-tolerance relative to specific proteins, such as, e.g., the fusion of TAAs with specific foreign protein sequences (U.S. Pat. No. 5,869,057, U.S. Pat. No. 5,843,648 and U.S. Pat. No. 6,069,242), or via the respective presentation by antigen-presenting cells (Immunobiol. (1999), 201:1).

It is the object of the present invention to avoid the drawbacks of the tumor vaccines described in the prior art, and to provide an improved tumor vaccine that causes an efficient immune response against tumor cells.

In accordance with the invention, this object is achieved by an immunogenic antibody that comprises at least two different epitopes of a tumor-associated antigen. Preferably, the immunogenic antibody has a defined specificity and is, in particular, a monoclonal antibody and/or at least partially synthesized.

The term “immunogenic” is meant to encompass any structure that leads to an immune response in a specific host system. A murine antibody, or fragments of this antibody, will, for instance, exhibit a very strong immunogenic action in human organisms, which action will be further enhanced by a combination with adjuvants.

An immunogenic antibody according to the present invention is able to act immunogenically through its specificity or its structure. In a preferred manner, the immunogenic antibody according to the invention is able to induce immunogenity even in the denatured state or as a conjugate with selected structures or carrier substances.

The term “epitope” defines that region within a molecule, which can be recognized by a specific antibody, or which induces the formation of specific antibodies. Epitopes may be conformation epitopes or linear epitopes.

Epitopes, above all, imitate or comprise domains of a natural, homologous or derivatized TAA. These are comparable to TAAs at least by their primary structures and, possibly, secondary structures. Yet, epitopes may also completely differ from TAAs in this respect and imitate components of TAAs, primarily proteinic or carbohydrate antigens, merely by the similarity of spatial (tertiary) structures. Thus, the tertiary structure alone, of a molecule is able to form a mimicry (“immunological imitation”; such as disclosed, e.g., in WO 00/73430) which induces an immune response to a specific TAA.

As a rule, it is to be anticipated that by an antigen which imitates a proteinic epitope of a tumor-associates antigen, a polypeptide of at least five amino acids is to be understood.

The epitopes of the antibody according to the invention preferably include at least one epitope of an antigen selected from the group consisting of peptides or proteins, particularly EpCAM, NCAM, CEA and T-cell peptides preferably derived from tumor-associated antigens, furthermore of carbohydrates, particularly Lewis Y, sialylTn, GloboH, and of glycolipids, particularly GD2, GD3 and GM2. Preferred epitopes are derived from antigens that are specific for epithelial tumors and occur to an increasing extent, for instance, in breast cancer, cancer of the stomach and intestines, prostatic cancer, pancreatic cancer, ovarial cancer and lung cancer. Among the preferred epitopes are those which cause above all a humoral immune response, i.e., a specific antibody formation in vivo. The immunogenic antibody according to the invention is preferably also able to trigger a T-cell-specific immune response, whereby not only antibodies of, for instance, the IgM class, but also antibodies of the IgG class will be formed in reaction to the administration of said antibody.

Alternatively, also those antigens which generate T-cell-specific immune responses may, in particular, be selected as epitopes in the sense of the invention. Among those, also intracellular structures or T-cell peptides are to be found above all.

Further preferred proteinic epitopes that are especially expressed on cancer cells of solid tumors include, e.g., TAG-72, MUC1, folate binding protein A-33, CA125, HER-2/neu, EGF-receptors, PSA, MART etc. (cf., e.g., Sem. Cancer Biol. 6 (1995), 321). Furthermore, also so-called T-cell epitope peptides (Cancer Metastasis Rev. 18 (1999), 143; Curr. Opin. Biotechnol. 8 (1997), 442; Curr. Opin. Immunol. 8 (1996), 651) or mimotopes of such T-cell epitopes (Curr. Opin. Immunol. 11 (1999), 219; Nat. Biotechnol. 16 (1998), 276-280) may be envisaged. Suitable epitopes are expressed in at least 20%, preferably at least 30%, of the incidences of tumor cells of a specific type of cancer, even more preferred in at least 40% and, in particular, at least 50% of the patients.

Carbohydrate epitopes preferred according to the invention are tumor-associated carbohydrate structures like the Lewis antigens, e.g., Lewis X, Lewis B and Lewis Y structures, as well as sialylated Lewis X structures. In addition, also GloboH structures, KH1, Tn antigen, in a particularly preferred manner sialylTn antigen, TF antigen, alpha-1-3,galactosyl epitope are preferred carbohydrate antigen structures encompassed by the present invention.

In a particular embodiment, at least two identical or different epitopes of an adhesion protein, for instance, a homophilic cellular membrane protein, such as EpCAM, are provided or mimicked on the antibody according to the invention. Thus, a plurality of antibodies having a specificity for the same molecule, yet different EpCAM binding sites can be generated by active immunization.

The antibody according to the invention may, however, also be made available in the form of a glycosilated antibody, the glycosylation itself being also able to imitate an epitope of a carbohydrate epitope of a TAA.

In a particular embodiment, at least two different epitopes are provided or imitated, at least one epitope being derived from the group of peptides or proteins and at least one epitope being derived from the group of carbohydrates. An epitope of an EpCAM protein and an epitope of a carbohydrate component, for instance of Lewis Y, or sialylTn, have turned out to be combined in a preferred manner. A Lewis Y-glycosylated antibody having a specificity for an EpCAM structure, in particular, constitutes an especially good immunogen in a vaccine formulation. This antibody is particularly able to imitate cellular tumor antigens, thus inducing the desired immune response to inhibit epithelial tumor cells.

In a preferred manner, the immunogenic antibody according to the invention acts as an antigen carrier, for instance of a proteinic antigen, in the vaccine. This means that the antibody according to the invention constitutes a multivalent antigen, for instance a bi-, tri- or polyvalent antigen. The epitopes are presented in a manner to cause the vaccine to initiate an immune response against these epitopes. A vaccine containing an antibody in the form of a di-, tri- or polyvalent antigen is thus provided.

The antibody according to the invention is primarily used for active immunization and, therefore, administered in small quantities only. Thus, no particular side-effects are to be expected even if the antibody according to the invention is derived from a non-human species such as, for instance, a murine antibody. It is, however, assumed that a recombinant, chimeric as well as a humanized or human antibody combined with murine and human components are particularly safe for the administration in man. On the other hand, a murine portion contained in the antibody according to the invention is able to additionally provoke the immune response in man on account of its foreignness.

A preferred primary function of the immunogenic antibody according to the invention is the presentation of epitopes. The specific recognition of the tumor-associated antigen(s) whose epitopes it comprises is not necessarily required, yet it is additionally able to specifically bind to an epitope and, at the same time, present an epitope.

Although an antibody according to the invention may, of course, be derived from a native antibody optionally isolated from an organism or patient, an antibody derivative preferably selected from the group consisting of antibody fragments, conjugates or homologs, yet also complexes and adsorbates is usually employed. In any event, it is preferred that the antibody derivative contains at least portions of the Fab fragment, preferably along with at least parts of the F(ab′)₂ fragment, and/or parts of the hinge region and/or of the Fc part of a lambda or kappa antibody.

Furthermore, a single-chain antibody derivative such as, for instance, a so-called single chain antibody may also be used as an epitope carrier in the context of the invention. The antibody according to the invention is preferably of the type of an immunoglobulin like IgG, IgM or IgA.

On the antibody according to the invention, other substances such as peptides, glycopeptides, carbohydrates, lipids or nucleic acids, yet also ionic groups such as phosphate groups, or even carrier molecules such as polyethylene glycol or KLH may be additionally contained in a covalent manner in the molecule structure. These side groups themselves may possibly represent epitopes of tumor-associated antigens in the sense of the present invention.

It is preferred according to the invention to provide a monoclonal antibody which, as ab1, comprises itself a specificity for a TAA so as to be possibly able to bind directly to a tumor cell or its derivative. This is to appropriately localize an immune response, optionally on the site of a tumor or disseminated tumor cell. The specificity of the antibody is preferably likewise selected from the above-mentioned groups of TAAs and, in particular, from the group consisting of EpCAM, NCAM, CEA, Lewis Y and sialylTn antigens.

A particularly good immunogen for EpCAM is, for instance, an anti-EpCAM antibody that imitates or comprises at least one or at least two EpCAM epitopes, for instance by its EpCAM-similar idiotype. Such an antibody is, for instance, derived from an anti-EpCAM antibody from WO 00/41722.

In an alternative embodiment, the antibody according to the invention may, however, also be selected so as to specifically bind an antibody. In the tumor vaccine according to the invention, especially anti-idiotypical antibodies, i.e. ab2, are preferably used for active immunization. These antibodies may be equipped with additional sequences or structures in order to obtain an immunogen according to the invention. Anti-idiotypical antibodies according to the invention preferably recognize again the idiotype of an antibody directed against a TAA. Thus, an epitope of a TAA is already formed on the paratope of the anti-idiotypical antibody as a mimicry for the TAA. The selection of the epitopes is again preferably made from the above-mentioned TAA groups. As an example, an anti-idiotypical antibody is used against glycan-specific antibodies, for instance, an anti-idiotypical antibody recognizing the idiotype of an anti-Lewis Y antibody, e.g. as described in EP 0 644 947.

The immunogenic antibody according to the invention is, above all, suitable as a basis for pharmaceutical preparations and, in particular, vaccines. Preferred are pharmaceutical preparations containing pharmaceutically acceptable carriers. These include, for instance, adjuvants, buffers, salts, preservatives. These pharmaceutical preparations may, for instance, be used for the prophylaxis and therapy of cancer-associated pathological conditions such as the metastasization in cancer patients. To this end, antigen-presenting cells are specifically modulated in vivo or also ex vivo, in order to generate an immune response to the TAAs comprised by the immunogenic antibody.

A vaccine formulation preferred in accordance with the invention mostly contains the immunogenic antibody only in small concentrations, for instance in an immunogenic quantity ranging from 0.01 μg to 10 mg. Depending on the nature of the antibody, whether by species-foreign sequences or by derivatization, yet also on the auxiliary agents or adjuvants employed, the suitable immunogenic dose is selected to range approximately from 0.01 μg to 750 μg and, preferably, from 100 μg to 500 μg. A depot vaccine to be released to the organism over an extended period of time may, however, also contain far larger antibody quantities such as, for instance, at least 1 mg to more than 10 mg. The concentration is a function of the amount of liquid or suspended vaccine administered. A vaccine is usually provided in ready-to-use syringes having volumes of from 0.01 to 1 ml, preferably 0.1 to 0,75 ml. These are, in fact, concentrated solutions or suspensions.

The immunogenic antibody in the vaccine according to the invention is preferably presented in a pharmaceutically acceptable carrier suitable for subcutaneous, intramuscular, but also intradermal or transdermal administration. Another mode of administration functions via the mucosal pathway, for instance, the vaccination by nasal or peroral administration. If solids are used as adjuvants for the vaccine formulation, an adsorbate or a suspended mixture of the antibody with the adjuvants is, for instance, applied. In special embodiments, the vaccine is administered as a solution or a liquid vaccine contained in an aqueous solvent.

Vaccine units of the tumor vaccines are preferably provided in suitable ready-to-use syringes. Since an antibody is relatively stable as compared to TAAs, the vaccine according to the invention offers the essential advantage of being marketable as a storage-stable solution or suspension already in a ready-to-use form. A content of a preservative like thimerosal or any other preservative with improved tolerance is not necessarily required, yet may be provided in the formulation to extend storage life at storage temperatures from refrigerator temperature to room temperature. The vaccine according to the invention may, however, also be provided in frozen or lyophilized form to be thawed or reconstituted on demand.

In any event, it has proved successful to enhance the immunogenity of the antibody according to the invention by the use of adjuvants. To this end, vaccine adjuvants such as, for instance, aluminum hydroxide (Alu gel) or phosphate, e.g. growth factors, lymphokins, cytokins such as IL-2, IL-12, GM-CSF, gamma interferon, or complementary factors such as C3d and, furthermore, liposome preparations or lipopolysaccharide from E. coli (LPS), yet also formulations with additional antigens against which the immune system has already induced strong immune responses, such as tetanus toxoid, bacterial toxins like Pseudomonas exotoxins and derivatives of lipid A.

To formulate vaccines, also other known methods for conjugating or denaturizing vaccine components may be employed in order to further enhance the immunogenity of the active substance.

Particular embodiments of the vaccine according to the invention contain additional vaccination antigens, particularly anti-idiotypical antibodies, i.e., mixtures of the immunogenic antibody according to the invention with various antibodies that are administered at the same time.

The immunogenic antibody according to the invention is also suitable for the preparation of diagnostic agents according to the invention. Thus, reagents containing the immunogenic antibody in association with other reactants or detection agents may be offered as diagnostic agents in set form. Such an agent preferably contains a label for the immediate detection of the antibody or its reaction product. The diagnostic agent according to the invention is, for instance, used for the qualitative and/or quantitative assessment of tumor cells or metastases or the determination of a metastasizing potential, said agent acting by an immune reaction or immune complexation.

The immunogenic antibody can be produced by a method according to the invention comprising the steps of:
a) providing an antibody; and
b) coupling at least two epitopes of a tumor-associated antigen to said antibody.

Alternatively, the method according to the invention may already depart from an anti-idiotypical antibody, the method steps in that case comprising:
a) providing an antibody including the idiotype of a tumor-associated antigen; and
b) coupling at least one epitope of a tumor-associated antigen to said antibody.

Coupling is usually effected by chemical or biological, e.g. enzymatic, reactions. The connection of an antibody with an epitope is, however, also feasible already on a molecular biological level. A conjugated product can be expressed and prepared just by the recombination of nucleic acids. Such methods according to the invention are characterized by the steps of:
a) providing a nucleic acid encoding an antibody including the idiotype of a tumor-associated antigen; and
b) recombining said nucleic acid with a nucleic acid encoding an epitope of a tumor-associated antigen or its mimicry; or
a) providing a nucleic acid encoding an antibody; and
b) recombining said nucleic acid with one or several nucleic acid(s) encoding at least two epitopes of a tumor-associated antigen or its mimicry.

The antibody, on which the invention is based, may, for instance, be an anti-idiotypical antibody, i.e. an ab2, and/or an antibody having a specificity for a tumor-associated antigen, i.e. an ab1.

The coupling corresponds to a conjugation for the formation of a covalent bond. A derivative that differs from native antibodies will, thus, be synthesized.

The combination according to the invention, of two immunogenic TAA mimicries completely different in nature in a surprising manner allows for an extremely efficient immunization against tumor-associated or tumor-specific structures such that the endogenous immune system will be efficiently protected against the respective tumors or able to combat these tumors.

The antibody according to the invention functions as a proteinic antigen-carrier which is present, for instance, with a carbohydrate antigen to constitute a conjugate of the invention. It is likewise feasible to provide several carbohydrate antigens in the conjugate according to the invention. Thus, several different glycans triggering immune responses against two or several different tumor-associated carbohydrate structures may, for instance, be coupled to one antibody. Such a conjugate does not occur in natural systems. The autoantigenic structures are thereby recognized as foreign, which will additionally intensify immunogenity. In accordance with the invention, a conjugate of this type is, therefore, present in a synthetic constellation naturally occurring neither sterically nor functionally (i.e., in tumor cells).

The coupling according to the invention to a molecule, of two structures completely different in nature, in addition to the advantage of a simple formulation of the synthetic vaccine also results in a much simpler vaccination scheme, since the same vaccine can always be used: Both the initial vaccination and also subsequent booster vaccinations are preferably given using the same vaccine.

Moreover, the invention relates to a method for immunogenizing epitopes of tumor-associated antigens or their mimicries. To this end, primarily low-molecular epitopes of the antigens are used, which by themselves would hardly be recognized by the immune system of mammals, particularly man. Immunogenization is effected in a manner that an antigen is conjugated to an antibody, with the antibody functioning as a carrier. By the method according to the invention, it is feasible to render immunogenic a plurality of epitopes and naturally, in particular, the epitopes of the already mentioned selection of antigens. The immunogenic antibody produced according to the invention preferably contains the epitope to be immunogenized and a further epitope of a tumor-associated antigen.

Immunogenization yields a material that is surprisingly well apt for the immunization of patients. The product to be obtained by the invention is, therefore, preferably provided as a vaccine.

Methods for detecting suitable antigenic structures, modelling and preparing TAA-derived peptides, polypeptides or proteins, or nucleic acids encoding the same, and, furthermore, lipoproteins, glycolipids, carbohydrates or lipids are known to the skilled artisan and can be provided for the respective tumor-specific structure without too much of an experimental expenditure. Furthermore, methods for conjugating proteins with such structures are known, which are suitable for the method according to the invention.

The carbohydrate structures selected as epitope mimicries can be derived from natural or synthetic sources, the carbohydrates being present as glycoproteins or glycolipids and capable of being coupled as such to the respective carrier molecule.

Also the antibody components can be chemically synthesized and subsequently connected with epitope structures, or synthesized together. By the chemical synthesis of antibody carrier molecules, it is feasible to introduce reactive groups on particular sites in order to be able to control both the extent of coupling with an epitope and the type and location of the bond.

The antibody carriers can also be produced as recombinant molecules by genetic engineering. It is conceivable to produce these antibodies in host cells that do not effect glycosylation (such as, e.g., Escherichia coli). Such polypeptides may then be chemically or enzymatically coupled to a desired carbohydrate antigen.

It is, however, also conceivable that the antibody carrier is produced in cells that are able to glycosylate the molecule. The genetic modification of nucleic acids encoding native antibodies may, for instance, cause the formation of appropriate glycosylation sites in the translated molecule.

The glycosylation of such a recombinant gene product with the respective tumor-associated glycan structures can be effected by production in cells genetically modified to appropriately glycosylate proteins. Such cells may be natural isolates (cell clones) than can be found by adequate screening for the desired glycosylation.

It is, however, also feasible to modify cells in a manner that they will express the respective enzymes necessary for the desired glycosylation, such that the desired glycosylation on the recombinant polypeptide carrier protein will be exactly found (Glycoconj. J. (1999), 16:81).

It is, however, also feasible to enzymatically produce, or modify, the glycosylation patterns of proteins (Clin. Chem. Lab. Med. (1998), 36:373).

In the immunogenic antibody according to the invention, the various epitope structures may be coupled to one another via a coupler. Such a coupler is preferably comprised of a short, bifunctional molecule such as, e.g., N-hydroxysuccinimide. Coupling via nitrophenyl-activated sugars is feasible too. In a preferred embodiment, coupling is effected via sulfhydryl groups (Biochim. Biophys. Acta (1983), 761, 152-162). Examples of sulfhydryl-reactive linkers are BMH, DFDNB or DPDPB. Yet, the coupler may also be realized by a larger chemical compound than a simple coupler molecule. The prerequisite always being that such a coupler will not adversely affect the immunological properties of the conjugate, i.e., will not itself trigger any substantial immunogenity. According to the invention, a coupler may also be produced quasi-“in situ” by the chemical conversion of a portion of the antibody or the structure to be conjugated. This coupler produced on the antibody or epitope structure itself can then be directly conjugated to the respectively other binding partner (e.g., via the amine group of lysine, via OH groups, sulfur groups, etc.). Coupling methods are known from the prior art (Anal. Biochem, (1986) 156, 220-222; Proc. Natl. Acad. Sci., (1981), 78, 2086-2089; Biochem. Biophys. Res. Comm. (1983), 115, 29-37).

According to a particular embodiment of the present invention, the antibody according to the invention comprises a nucleic acid molecule encoding a proteinic TAA as an epitope structure in the sense of the present invention, said nucleic acid being covalently conjugated.

The present invention also relates to a set suitable for tumor vaccination. The set comprises a preparation of an immunogenic antibody according to the invention and a suitable application means such as, e.g., syringes, infusion devices, etc. If the conjugate preparation is present in lyophilized form, the set will further comprise a suitable reconstitution solution optionally including special stabilizers or reconstitution accelerators.

The present invention, by which the immunogenic antibody including several different epitope structures and, in particular, the structure of a tumor-associated carbohydrate antigen is provided, enables the triggering of an immune response having two or more specificities and, thus, combatting a tumor cell by two or more different tumor-associated antigens. As a result, the effective range of the vaccine is widened and more specifically designed.

The invention will be explained in more detail by way of the following examples and the figures of the drawing, yet without being imited thereto.

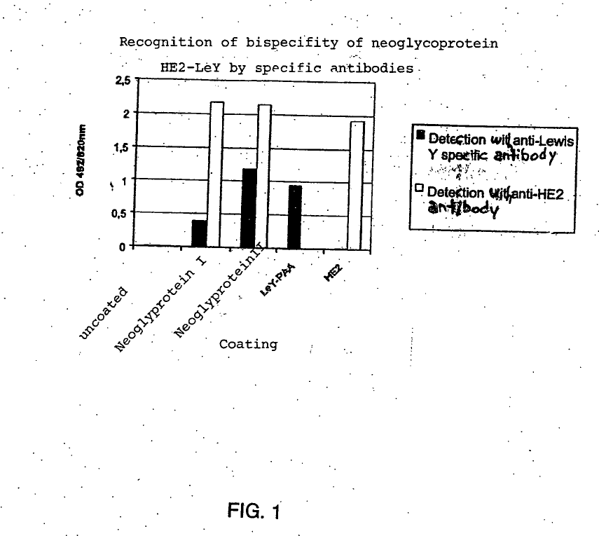

FIG. 1 illustrates the recognition of the bispecificity of the neoglycoprotein HE2-LeY by specific antibodies;

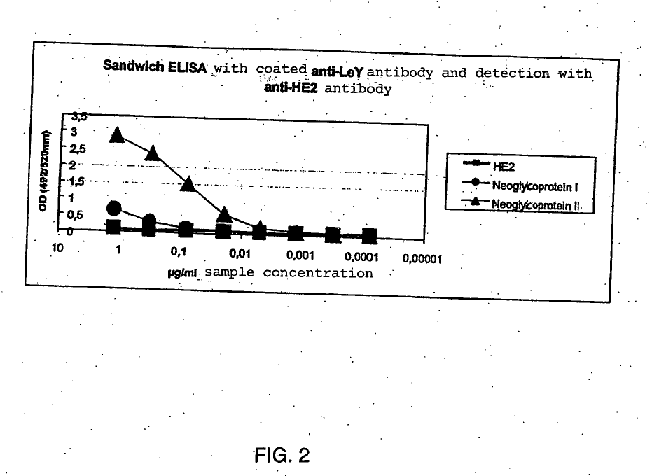

FIG. 2 shows a sandwich ELISA using coated anti-LeY antibody and dectection with anti-HE2 antibody;

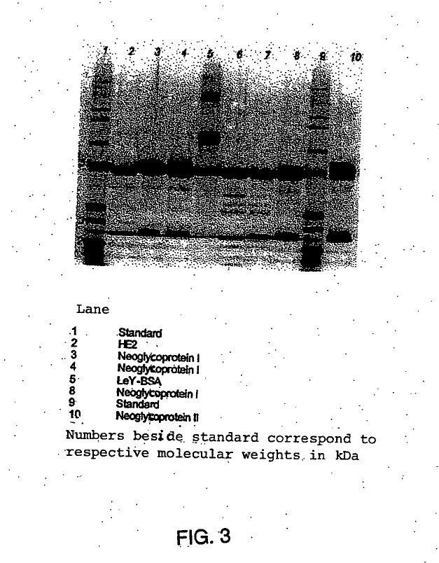

FIG. 3 depicts the SDS-PAGE of different neoglycoconjugates;

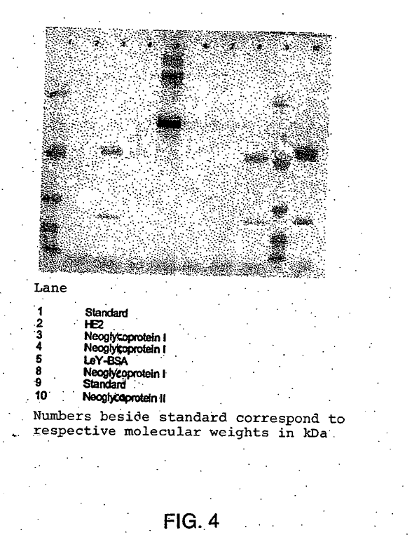

FIG. 4 illustrates an immuno-Western blot of the SDS-PAGE of different neoglycoconjugates;

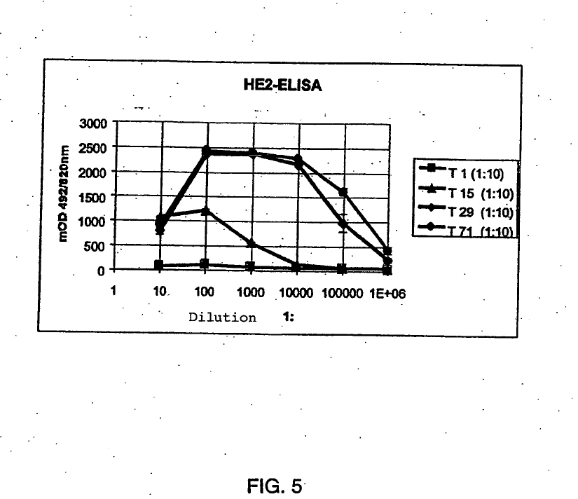

FIG. 5 shows a HE2-ELISA; and

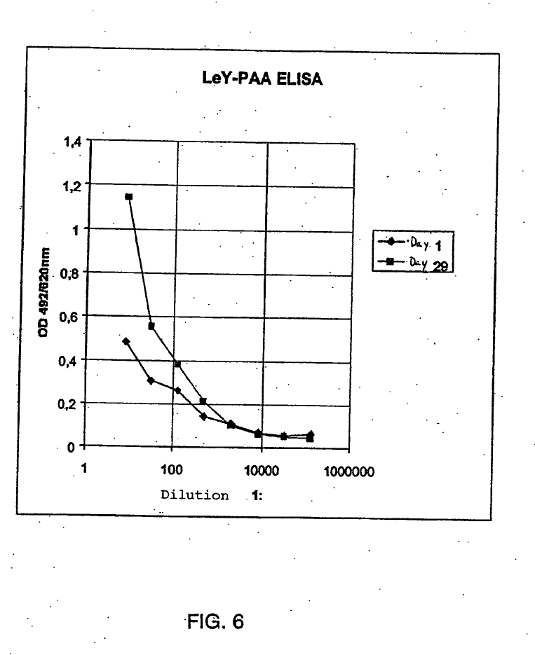

FIG. 6 shows a LeY-PAA-ELISA.

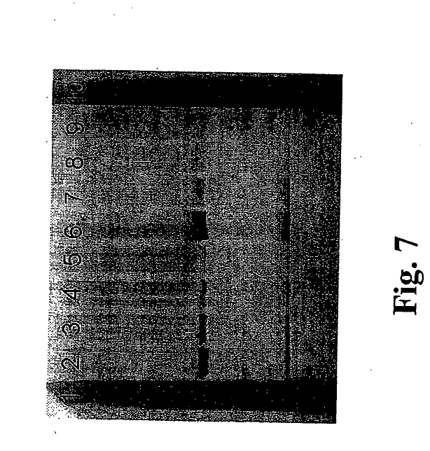

FIG. 7 shows an LDS PAGE. From a comparison of HE2 (lanes 2-5) with the HE2-sialylTn coupling product (lanes 6-8), a clear rise in the molecular weight of the heavy chain is apparent. This means that sialylTn has been successfully coupled to the heavy chain (50 kDa) of the HE2 antibody. Moreover, the occurrence of a second band (of slightly higher molecular weight) in addition to the 25 kDA band indicates that sialylTn too has been partially coupled to the light chain too.

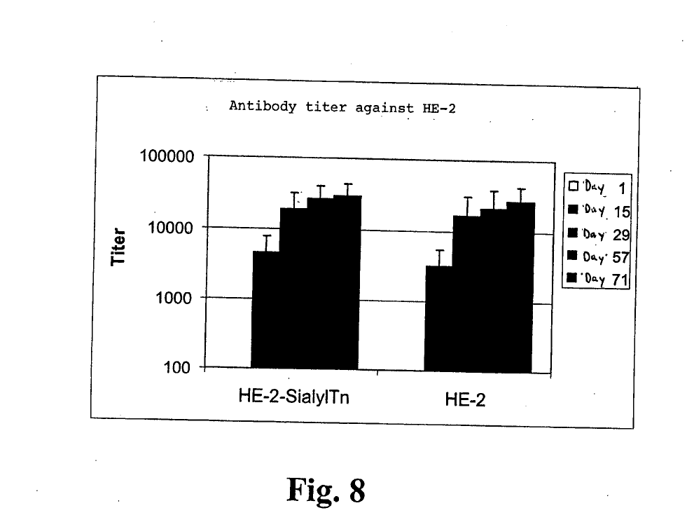

FIG. 8 shows the antibody titer against HE2. The induction of the immune response to HE2 by the HE2-sialylTn multi-epitope vaccine is comparable to that induced by HE2.

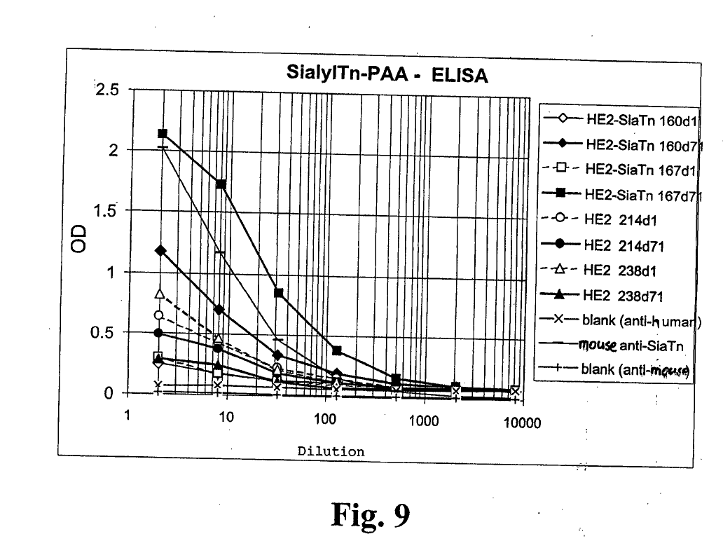

FIG. 9 shows sialylTn-PAA ELISA.

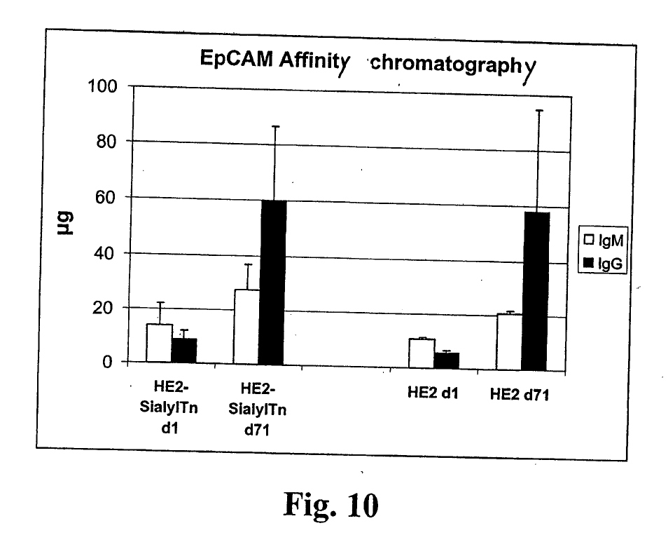

FIG. 10 shows EpCAM affinity chromatography. The results indicate that the binding of the HE2-sialylTn-vaccine-induced antibodies against EpCAM in the serum after immunization is comparable to that of HE2.

### EXAMPLES

### Example 1

### Coupling of a Lewis Y Carbohydrate to an EpCAM-Specific Antibody

The antibody HE2 is described in the patent application WO 00/41722 and upon an appropriate immunization is able to induce an immune response binding to tumor cells. According to the invention, a synthesized Lewis Y carbohydrate antigen is coupled to HE2. In this example, coupling is effected chemically:

The antibody HE2 is coupled to N-hydroxysuccinimide-activated synthetic Lewis Y tetrasaccharide (Syntesome GmbH, Munich, Germany) in a suitable buffer (100 mM sodium phosphate buffer containing 150 mM NaCl, pH 8.5).

N-hydroxysuccinimide-activated Lewis Y-tetrasaccharide is dissolved in N,N-dimethylformamide (100 mg/ml) and added drop-wise to an HE2 antibody solution in the appropriate buffer (100 mM sodium phosphate puffer containing 150 mM NaCl, pH 8.5) and shaken for at least 2.5 hours at 4° C. The extent of glycosylation of the antibody with Lewis Y can be controlled by selecting the molar excess of activated sugar as well as the concentration of antibody-containing solution (1-10 mg/ml). For comparative purposes, two different reaction batches are prepared by varying the molar excess (5-fold and 15-fold, respectively) of activated sugar: “neoglycoprotein I” having a lower carbohydrate portion and “neoglycoprotein II” having a higher carbohydrate portion.

The bispecificity of the neoglycoprotein can be detected by various immunological methods (ELISA or Western blotting with antibodies directed against the Lewis Y determinant or against HE2).

Direct ELISA:

HE2, HE2-Lewis Y-neoglycoprotein or LeY-PM (polyacrylamide-coupled tetrasaccharide, Syntesome 045-PA) is dissolved in a coating buffer (15 mM Na₂CO₃, 5 mM NaHCO₃, 3 mM NaN₃, pH 9.6) (10 g/ml) and bound to a microtiter plate (Nunc, Denmark, Maxisorb) (1 hour at 37° C., 100 μl/well). After three-time washing of the microtiter plates with washing buffer (2% NaCl, 0.2% Triton X-100 in PBS; 2001) blocking is effected with 5% fetal bovine serum in PBS (138 mM NaCl, 1.5 mM KOH, 2.7 mM KCl, 6.5 mM Na₂HPO₄, pH 7.2; 200 μl) (30 minutes at 37° C.) and subsequently—after repeated washing—incubation with specific anti-Lewis Y antibody (human) or goat anti-HE2 antibody (1 μg/ml dissolved in dilution buffer: 2% FCS in PBS; 100 μl) was effected for half an hour at 370° C. Unbound antibodies are removed by three-time washing with washing buffer. The bound antibodies are detected by an HRP conjugate specific for the respective detection antibody (goat anti-human IgG+A+M HRP of Zymed (USA) for anti-Lewis Y antibody; mouse anti-goat IgG HRP (Axell, USA) for anti-HE2 antibody, 1 μg/ml, 1001) (30 minutes at 37° C.). After subsequent washing (3× with washing buffer and 1× with staining buffer), the staining of 100 μl orthophenylene diamine dihydrochloride solution (Sigma, USA; dissolved in staining buffer and activated with H₂O₂; 30%, Merck, Germany) is initiated by bound HRP conjugate and the color development is stopped with 15% sulfuric acid (50 μl). On a microplate photometer (Labsystem, Model No. 354), the developed extinction is measured at 492 nm, the reference wavelength being 620 nm.

After a further washing step with staining buffer (24.3 mM citric acid, 51.4 mM Na₂HPO₄, pH5).

In FIG. 1, the results are illustrated: Both of the two neoglycoproteins exhibit both specificities (HE2 and Lewis Y), neoglycoprotein II being more strongly functionally glycosylated than neoglycoprotein I and, therefore, emitting a higher signal with the anti-Lewis Y antibody.

Sandwich ELISA:

Human anti-Lewis Y antibody (10 μl/ml dissolved in coating buffer; 1001) is nonspecifically bound to a microtiter plate (Nunc, Maxisorb) (1 hour incubation at 37° C.), after three-time washing with washing buffer (2001) is blocked with 5% fetal bovine serum in PBS (200 μl) (incubation for 30 minutes at 37° C.) and incubated with HE2-Lewis Y-neoglycoproteins I and II as well as HE2 as a control in various concentrations (1.25-7.63×10⁻⁶ μg/ml; 100 μl) for 1 hour at 37° C. After three-time washing in washing buffer, incubation is effected with goat anti-HE2 antibody (1 μg/ml in dilution buffer; 100 μl) for 30 minutes at 37° C. Excess antibodies are removed in a subsequent washing step (3× with washing buffer). Bound antibodies are recognized by incubation (30 minutes, 37° C.) with mouse anti-goat IgG HRP (Axell, dissolved 1:1000 in dilution buffer, 100 μl): After subsequent washing (3× with washing buffer, 1× with staining buffer), bound HRP conjugate triggers a staining reaction of 1001 added orthophenylene diamine dihydrochloride solution (Sigma, 10 mg dissolved in 25 ml staining buffer and activated with 10 μl H₂O₂; 30%, Merck). The color reaction is stopped with 50 μl 15% H₂SO₄ and the extinction is measured at 492 nm (reference wavelength 620 nm) on a microplate photometer (Labsystem, Model No. 354).

From FIG. 2 it is apparent that both of the two neoglycoproteins can be detected in this sandwich ELISA, “neoglycoprotein II” being more strongly glycosylated and, therefore, more strongly retained by the precoated anti-Lewis Y antibody.

SDS-PAGE:

The samples (nonconjugated HE2 antibody, neoglycoproteins I and II as well as Lewis Y-BSA) are heat-treated in reducing buffer (85° C., 2 minutes) and electrophoretically separated on polyacrylamide gel (4-12% Bis-Tris Gel). The proteins thus separated according to size are visualized by silver staining (NOVEX SDS-PAGE-System, Invitrogen, USA). On the gel, only a very slight increase in the molecular weight due to glycosylation with Lewis Y tetrasaccharide is to be noted (cf. FIG. 3).

Western Blot:

As with SDS-PAGE, the samples are separated according to size. After this, the separated proteins are blotted on a nitrocellulose membrane, blocked for an hour in 3% milk powder solution and subsequently incubated with human anti-Lewis Y antibody (10 μg/ml in PBS) for two hours. Bound antibodies are detected by goat anti-human HRP conjugate (1:500 in PBS) specific for anti-Lewis Y antibody. The Lewis Y glycosylated proteins are visualized by a subsequent color reaction.

As is apparent from FIG. 4, neoglycoprotein II reacts with anti-Lewis Y antibody; neoglycoprotein I appears to have been glycosylated too weakly to be detected by anti-Lewis Y in this assay.

Immune Response to HE2 and Lewis Y:

Sera of immunized monkeys are examined for the formation of humoral immune responses to HE2 and Lewis Y at different times before and after immunization. The immunization scheme is as follows (the individual immunizations being performed subcutaneously: 500 μg protein adsorbed on 1.67 mg aluminium hydroxide in 0.5 ml 1 mM phosphate buffer pH 6.9/155 mM NaCl).

Times of Immunization:

Day 1 (T1)
Day 15 (T15)
Day 29 (T29)
Day 43 (T43)
Day 57 (T57)
Day 71 (T71)
Blood Collections for Serum Isolation:
Day 1 (T1)
Day 15 (T15)
Day 29 (T29)
Day 43 (T43)
Day 57 (T57)
Day 71 (T71)
Day 92 (T92)

HE2-ELISA:

HE2 antibody solution is diluted to 10 μg/ml in coating buffer and incubated at 37° C. for 1 hour (100 μl). After three-time washing with washing buffer, blocking is effected with 200 μl 5% fetal bovine serum in PBS for 30 minutes at 37° C. After a further washing step (as previously described), 100 μl of different dilutions of the sera of immunized animals are applied on a microtiter plate (dilution buffer: 2% fetal bovine serum in PBS) and incubated for 1 hour at 37° C. Unbound antibodies are removed by three-time washing with washing buffer and subsequently incubated with 100 μl goat anti-human IgG+A+M HRP solution (Zymed, diluted 1:1000 in dilution buffer) for 30 minutes at 37° C. After three-time washing with washing buffer and one-time washing with staining buffer, a color reaction of orthophenylene diamine dihydrochloride (Sigma, 10 mg dissolved in 25 ml staining buffer activated with 10 μl H₂O₂, 30%, Merck) is triggered by bound HRP. The reaction is stopped with 50 μl sulfuric acid (15%, Fluka), and the extinction is measured at 492 nm (reference wavelength 620 nm) on a microplate photometer (Labsystems, Model No. 354).

FIG. 5 shows the result of the HE2-ELISA. It is apparent that the immune response against the carrier protein is very strong already after 2 immunizations.

By immunizing a Rhesus monkey with neoglycoprotein, a strong humoral immune response against HE2 is, thus, induced.

Lewis Y-AA ELISA:

Lewis Y-PAA (Lectinity Holding, Inc., Bad Homburg, Germany) is diluted to 10 g/ml in a coating buffer and incubated for 1 hour at 37° C. (1001). After three-time washing with washing buffer, blocking is effected with 200 μl 5% fetal bovine serum in PBS for 30 minutes at 37° C. After a further washing step (as previously described), 1001 of different dilutions of the sera of immunized animals are applied on the microtiter plate (dilution buffer: 2% fetal bovine serum in PBS) and incubated for 1 hour at 37° C. Unbound antibodies are removed by three-time washing with washing buffer and subsequently incubated with 1001 goat anti-human IgG+A+M HRP solution (Zymed, diluted 1:1000 in dilution buffer) for 30 minutes at 37° C. After three-time washing with washing buffer and one-time washing with staining buffer, a color reaction of orthophenylene diamine dihydrochloride (Sigma, 10 mg dissolved in 25 ml staining buffer activated with 10 μl H₂O₂, 30%, Merck) is triggered by bound HRP. The reaction is stopped with 501 sulfuric acid (15%, Fluka), and the extinction is measured at 492 nm (reference wavelength 620 nm) on a microplate photometer (Labsystems, Model No. 354).

FIG. 6 indicates that the immunization of a Rhesus monkey with neoglycoprotein triggers a humoral immune response specifically directed against Lewis Y.

### Example 2

### Coupling of the SialylTn Carbohydrate to HE2

SialylTn-O(CH₂)₃NH(CH₂)₄COO-pNp was coupled to HE2.

The final product was analyzed by means of SEC, LDS-PAGE, Western Blot and various ELISAs.

Methods

Material
HE2 Panorex, 10 mg/ml, Lot 170901
SialylTn-O(CH₂)₃NH(CH₂)₄COO-pNp, 2×5 mg; Lectinity
DMF (N,N-dimethylformamide (anhydrous, Merck))
Couplung buffer: 0.1M Na₂HPO₄+0.15M NaCl (pH=8)
Formulation buffer: NaCl 0.86%+1 mM Na₂HPO₄ (pH=6.0)

Methods
1. 100 mg HE2 (v=10 ml; conc: 10 mg/ml) were dialyzed against 2×700 ml coupling buffer, using a Slide-A-Lyzer Dialysis Cassette at 4° C. for 20 hours, filling up of the volume to −10 ml, the concentration according to SEC was −10 mg/ml.
2. 2×5 mg sialylTn-O(CH₂)₃NH(CH₂)₄COO-pNp were dissolved with 2×100 μl DMF (1001/tube).
3. The solution of SialylTn (in DMF) was filled up to ˜10 ml (˜100 mg) with ice-cooled HE2 (in coupling buffer).
4. Both of the sialylTn vials were washed with 1001 DMF (with a transfer from tube 1 to tube 2), this was also added to the reaction mixture.
5. The reaction mixture is allowed to rotate over night (28 hours) at +4° C. The reaction kinetics was examined by SEC.
6. The final solution of HE2-sialylTn (10 ml, ˜10 mg/ml) was dialyzed against 2×800 ml formulation buffer using a Slide-A-Lyzer Dialysis Cassette at 4° C. for 20 hours.

Analysis:

Size Exclusion Chromatography:

The concentrations of HE2-sialylTn were quantified by size exclusion chromatography (SEC) on a ZORBAX GF-250 column in a Dionex System. The HPLC system was tested with a gel filtration standard. (BioRAD).

HE2 was chosen as reference standard for the quantification of HE2-sialylTn. The decrease of the retention time (correlating with the increase in molecular weight) correlates with the effectiveness of the coupling reaction of sialylTn to He2. The data obtained show that the coupling efficiency increases with the reaction time and a saturation is reached at 23-27 hours.

LDS-PAGE (Lithium Dodecyl Sulfate PAGE)

LDS-PAGE with Bis-Tris Gel (4-12%)

SilverXpress™ staining: cf. “NuPAGE Bis-Tris Gel” Instruction Booklet, page 13

The results are indicated in FIG. 7.

| | | | Volume | |
|---|---|---|---|---|
| Lane | Sample | Conc. | [μl] | Preparation |
| 1 | Mark 12 MW Standard | — | 10 | none |
| 2 | HE2 dialyzed in coupling | 20 μg/ml | 10 | cf. SOP |
| | buffer | | | |
| 3 | HE2 dialyzed in coupling | 10 μg/ml | 10 | cf. SOP |
| | buffer | | | |
| 4 | HE2 dialyzed in coupling | 50 μg/ml | 10 | cf. SOP |
| | buffer | | | |
| 5 | HE2 dialyzed in coupling | 2.5 μg/ml | 10 | cf. SOP |
| | buffer | | | |
| 6 | HE2SiaTn dial. in formulation | 20 μg/ml | 10 | cf. SOP |
| | buffer | | | |
| 7 | HE2SiaTn dial. in formulation | 10 μg/ml | 10 | cf. SOP |
| | buffer | | | |
| 8 | HE2SiaTn dial. in formulation | 5 μg/ml | 10 | cf. SOP |
| | buffer | | | |
| 9 | HE2SiaTn dial. in formulation | 2.5 μg/ml | 10 | cf. SOP |
| | buffer | | | |
| 10 | Mark 12 MW Standard | — | 10 | none |

FIG. 7: As compared to HE2 (lanes 2-5), a marked increase in the molecular weight of the heavy chain occurs in the HE2-sialylTn coupling product (lanes 6-8), which indicates that sialylTn has been successfully coupled to the heavy chain (50 kDa) of the HE2 antibody. Moreover, the occurrence of a second band (having a slightly different molecular weight) in addition to the 25 kDA band indicates that even the light chain has been partially coupled to sialylTn.

Western Blot

Western Blot with Rabbit × Mouse IgG2a

Method:
1. LDS gel with BIS-Tris-Gel (4-12%)
2. Western transfer: instructions cf. NuPAGE Bis-Tris-Gel Instruction Booklet pages 14-20 (using Immobilon Transfer Membrane PVDF 0.45 μm, Millipore)
3. Membrane development:

Material:
Conjugate: rabbit × mouse IgG2a —HRP, #61-0220, Zymed
Staining solution 1: 15 mg HRP color reagent (BioRAD) in 5 ml MetOH
Staining solution 2: 15 μl 30% H₂O₂ in 25 ml PBS def. 1×

Method:
Membrane blocking with 3% milk powder in PBS for 1 hr at RT
Membrane washing with PBS
Incubating with conjugate (dilution 1:1000 in PBS) for 1 hr at
RT
Membrane washing with PBS
Developing with staining solutions 1+2 and stopping of coloration with water.

Western blot with anti-sialylTn CD175s (IgG type)/rat × mouse IgG1-HRP.

Method:
1. LDS gel with BIS-Tris-Gel (4-12%)
2. Western transfer: instructions cf. NuPAGE Bis-Tris-Gel Instruction Booklet pages 14-20 (using Immobilon Transfer Membran PVDF 0.45 μm, Millipore)
3. Membrane development:

Material:
Secondary antibody: anti-sialylTn CD175s (IgG type), 90 μg/ml, DAKO, Code No. M0899, Lot 089(601)
Conjugate: rat × mouse IgG1-HRP, Becton Dickinson, Mat. No. 559626, batch: 37205
3% milk powder in PBS deflx
Staining solution 1: 15 mg HRP color reagent (BioRAD) in 5 ml MetOH
Staining solution 2: 15 μl 30% H₂O₂ in 25 ml PBS

Method:
Membrane blocking with 3% milk powder in PBS for 1 hr at RT
Membrane washing with PBS
Incubating with secondary antibody (concentration 10 g/ml) v=5 ml, for 1 hr at RT
Membrane washing with PBS
Incubating with conjugate (dilution 1:1000 in PBS) for 1 hr at RT
Membrane washing with PBS
Developing with staining solutions 1+2 and stopping of the re action with water.

The increase in the molecular weight of the heavy chain of the HE2 antibody after coupling with sialylTn was confirmed by Western blotting and staining with rabbit anti-mouse IgG2a-HRP.

A standard ELISA was carried out to demonstrate how much of the anti-idiotypical binding activity (of HE2) had been retained in the coupling product.

Immobilized IGN111 binds anti-idiotypical HE2, which is recognized by anti-mouse IgG2a-HRP.

It was demonstrated that He2 was about 2 to 3 times more reactive than HE2-sialylTn, which meant that only a slight loss of binding capacity had occurred after coupling.

Another standard ELISA was carried out to detect sialylTn by a mouse anti-sialylTn antibody. To this end, the starting material HE2 and the coupling product HE2-sialylTn were immobilized. Anti-sialylTn (mouse IgG)/rat anti-mouse IgG1-HRP were used for the detection of sialylTn.

The results indicate that the HE2-sialylTn reaction product does actually carry sialylTn groups as against HE2 prior to coupling.

Summary:

SialylTn was successfully coupled to HE2 antibody. The coupling reaction involves an extended reaction kinetics time, saturation being reached after about 24 hours. SialylTn was primarily coupled to the heavy chain of HE2 antibody, whereas the light chain was only partially coupled with sialylTn.

The HE2-sialylTn coupling product retains the majority of the idiotypical specificity of HE2, the sialylTn portion of this neoglycoprotein being recognized by sialylTn-specific antibodies.

These results together clearly indicate that the antigenic epitopes of both portions, i.e. the HE2 protein portion and the sialylTn portion, are preserved in the multi-epitope vaccine. The endotoxin content is below the detection limit.

### Example 3

### Formulation of HE2-sialylTn Using Different Adjuvants

1. Formulation buffer (NaCl, Na₂HPO₄), thimerosal, alhydrogel
2. Formulation buffer, thimerosal, alhydrogel, LPS, E. coli (Sigma, No. L-4391).

### Example 4

### Results of the Immunization of Rhesus Monkeys with HE2-SialylTn Neoglycoprotein: Tolerance and Immunogenicity Studies

Immunization Scheme and Blood Collections

Rhesus monkeys were vaccinated four times by subcutaneous immunization with 500 μl of the vaccine (containing 500 μg HE2 adsorbed on alhydrogel in 1 mM Na phosphate buffer, pH=6.0, supplemented with 0.86% NaCl).

Blood was taken on days —3, 1, 8, 15, 29, 57 and 71. The blood was allowed to coagulate (SST clot activator tube (Vacutainer)); serum was transferred into Nunc tubes 1.8 ml (3754318).

| Day | Date | Immunization | Blood Collection |
|---|---|---|---|
| −3 | | no | yes |
| 1 | | 500 μl | yes, before imm. |
| 8 | | no | yes |
| 15 | | 500 μl | yes, before imm. |
| 29 | | 500 μl | yes, before imm. |
| 57 | | 500 μl | yes, before imm. |
| 71 | | | yes |

FIG. 8 shows the results of the immunization studies in Rhesus monkeys. The induction of the immune response to HE2 by the HE2-sialylTn multi-epitope vaccine is comparable to the immune response induced by HE2.

ELISA

Preserum (day 1) and immune serum (day 15, 29, 57, 71) were analyzed by HE2 ELISA and sialylTn ELISA in respect to their immune responses to the immunizing antigen (HE2). A goat anti-human IgGAM-HRP (Zymed, No. 62-8320, Lot 20571004) conjugate was used for the detection.

The SialylTn ELISA was performed similarly to the Lewis Y ELISA except for some modifications. To sum up, ELISA plates (F96 Maxisorp microtiter plates, NUNC), for a period of 2 hrs at 37° C., were coated with 20 μg/ml sialylTn-PAA (30% mol, Syntesome) diluted in coating buffer. After the washing step (three times, WBK diluted 1:10) the ELISA plates were blocked with 5% FCS in PBS (30 min, 37° C.), followed by a subsequent washing step. The samples (prediluted in 2% FCS) were incubated for 1 hr at 37° C. NAS (NA pool 25-07-01, Biotest) and PBS were used as negative controls. For the sialylTn ELISA, a mouse anti-sialylTn CD175s antibody (DAKO, Code No. M0899, Lot No. 039(601)) was used at an initial concentration of 20 μg/ml, which served as a positive control.

After another washing step, the plates were incubated at 37° C. for 30 min with a goat anti-human Ig (H+ L)-HRP conjugate (1:4000, SB, Southern Biotechnology Cat. No. 2010-05, Lot No. L262-S496L) or a mouse anti-human IgG (Fc)-HRP conjugate (1:1000, SB, Cat. No. 9040-05, Lot No. J560-NC21G) or a mouse anti-human IgM-HRP conjugate (1:1000, SB, Cat. No. 9020-05, Lot No. H018-WO89), respectively. A rabbit anti-mouse IgG1-HRP (Zymed, No. 61-0120, Lot No. 00761146) was used to detect the mouse anti-sialylTn antibody (positive control).

After the next washing step, the substrate OPD (1 OPD tablet dissolved in 25 ml staining buffer+10 μl 30% H₂O₂) was added. After 10 minutes the color reaction was stopped by the addition of 50 μl H₂O₂ (30%).

FIG. 9 shows the results of the sialylTn-PAA ELISA. The induction of the immune response to the immunizing antigen HE2 and the target antigen EpCAM is comparable to that of the original HE2 single-epitope vaccine. Thus, an induction of the immune response against the carbohydrate antigen sialylTn occurred, this immune response was not induced upon vaccination with an HE2 single-epitope vaccine.

Affinity Chromatography

An affinity chromatography was carried out on an AKTA explorer, Pharmacia FPLC system. One ml of the serum (preserum or immune serum) was diluted 1:10 with PBS 1x+0.2M NaCl, pH=7.2 (=buffer A). After the equilibration of the column, the diluted serum was packed on the chromatography column at a flow rate of 1 ml/min. Unbound sample was washed off with buffer A until the UV line (280 nm) was below 5 mAU. The elution of the bound sample was performed stepwise with glycine buffer pH=2.9 (=buffer B, elution buffer). The desired fractions were immediately neutralized with 1M NaHCO₃ and stabilized by the addition of sodium azide (final concentration: 0.02%).

The following affinity chromatography columns were used:
1. HE2-Sepharose: HE2 coupled to CH-Sepharose 4B column, Lot 20000905-070301 (SS LJ5/174)
2. EpCAM-Sepharose: EpCAM coupled to CH-Sepharose 4B column (IF LJ32/54+57)
3. HE2-SialylTn-Sepharose: HE2-SialylTn coupled to CH-Sepharose 4B column

The purification of the preserum or immune serum was carried out either by a) single-step affinity chromatography, or b) sequential affinity chromatography with the eluate of the first column applied on a second affinity chromatography column, or c) differential affinity chromatography with the effluent of the first column loaded on a second affinity chromatography column.

After the quantification of the amounts of immunoglobulins by SEC, the remaining serum eluates were stabilized by the addition of FCS (final concentration 2%) and stored at +4° C. The results are shown in FIG. 10.

Size Exclusion Chromatography:

The amounts of immunoglobulins (IgG, IgM) were quantified by size exclusion chromatography (SEC) on a ZORBAX GF-250 column in a DIONEX system. The HPLC system was tested by a gel filtration standard.

For the quantification of the amounts of immunoglobulins contained in the ÄKTA eluates, a standard curve of human IgG (Sandoglobulin) was prepared in a range of 1.95-25 μg/ml and used as a reference standard.

Cell Lines

WM9, SKBR5, KATOIII, HT29, and OVCAR3 cells were cultivated with 10% FCS and 1% penicillin/streptomycin in RPMI1640 medium supplemented with L-glutamine. CT26 and CT26-KSA (clone #21 Sp1-3; EpCAM-transfected CT26 cells) were allowed to grow in DMEM supplemented with 10% FCS, 1% nonessential amino acids, 1% sodium pyruvate, 1% vitamin, 1-glutamine and 1% penicillin/streptomycin.

FACS Analysis

Cells were harvested in PBS buffer containing 0.2 mg/ml EDTA. Cultivation medium was added to the detached cells, the latter are subsequently pelletized and washed twice in FACS buffer (PBS buffer supplemented with 2% FCS and 0.1% NaN₃). 10,000 cells were blocked on ice for 30 minutes with PBS containing 10% FCS and 0.1%—sodium azide. After having transferred the cells into FACS buffer, the cells were incubated on ice for an hour with the eluates derived from the affinity chromatographies of the preserum and immune serum, respectively. The following primary antibodies were used as positive controls: IGN311 (EN25.888) for the Lewis Y coloration, and HE2 and KS1/4 for the EpCAM coloration. The cells were washed twice in FACS buffer and incubated with the detection antibodies under protection from light for 30 minutes (sheep anti-human IgGAM-FITC (gamma- and light-chain-specific), Silenius, dilution 1:1000 or rabbit anti-mouse IgGAM F(ab)₂′-FITC Dako (gamma- and light-chain-specific), dilution 1:100, for the detection of the murine HE2 and KS1/4 antibodies. After three-time washing in FACS buffer, the fluorescence intensities (10,000 cells in 100 μl FACS buffer per analysis) were measured by a FACS Calibur System (Becton Dickinson).

Control staining with:
IGN3 μl (25 μg/ml), KS1/4 (1 μg/ml), HE2 (1 μg/ml), SKBR5, KatoIII, WM9, CT26 and CT26KSA cells.

Results

Immune Response Against Immunizing Antigens (HE2)

The preserum (day 1) or immune serum (days 15, 29, 57, 71) of all animals was analyzed by HE2 ELISA in respect to their immune responses against the immunizing antigen (HE2). A clear immunizing effect was observed in all vaccinated groups, the intensity of the immune response increasing as a function of time (and the number of immunizations). It was important that none of the adjuvants raised the HE2 titer or allowed the kinetics of the immune response to rise as compared to that of the control group, which had received the antigen without adjuvant (P6/01). The multi-epitope HE2-sialylTn vaccine (P2/01) induced an HE2 titer comparable to that of the HE2 vaccine (P6/01).

The results are apparent from FIG. 8, this being a HE2 Rhesus monkey study.

The HE2-sialylTn multi-epitope vaccine induced an immune response against the second antigen, sialylTn, in all of the immunized animals, as was detected by sialylTn ELISA, this effect having not been found after vaccination with the HE2 single-epitope vaccine.

Serum Purification by Direct EpCAM Affinity Chromatography

Immune sera (day 71) were analyzed by direct EpCAM affinity chromatography followed by size exclusion chromatography (SEC). In all inoculated groups, significant amounts of Ig (IgG and IgM) were found in the immune sera (60-87 μg Ig), this being substantially higher than the content of Ig in the presera (13-22 μg). Furthermore, an IgG switch was to be observed after inoculation, with elevated IgG/IgM ratios in the immune serum. By contrast, the adjuvants did not cause any increase in Ig (IgG, IgM), which exhibited a specific reactivity with rEpCAM in the immune sera as assumed by direct EpCAM chromatography, in comparison to the HE2 control group.

SialylTn ELISA

The preserum (day 1) and immune serum (day 71) of the multi-epitope vaccine (P2/01, HE2-silalylTn) group in comparison to the P6/01 control group were assayed by sialylTn ELISA for their immune responses to the sialylTn carbohydrate antigen.

A marked immunization effect, i.e., the induction of the anti-sialylTn antibody titer, was found in all of the four immunized animals of the P2/01 group, by contrast no increase in the sialylTn antibody titer occurred in the HE2 control group after immunization.

The results are shown in FIG. 8, the induction of the immune responses to the immunized antigen (HE2) and the target antigen (EpCAM) is similar to that of the HE2 single-epitope vaccine. The immune response against the carbohydrate antigen sialylTn is induced by the multi-epitope vaccine, such induction being not observed after immunization with the HE2 single-epitope vaccine (P6/01).

### Example 5

### Preparation of a Recombinant Mouse IfG2a-HE2 Antibody (rHE2)

Molecular Biological Constructs

The bicistronic PIRES expression vector of Clontech Laboratories Inc., Palo Alto, USA, allows the expression of two genes on a high level and enables the translation of two consecutive open reading frames from the messenger RNA. In order to select positive transformants using a reporter gene, the internal ribosome entry site (IRES) was truncated in this expression vector, thus enabling lower expression rates to occur in this second reading frame.

In order to achieve this, the original IRES sequence had to be reestablished to enable our demands for the expression of the heavy and light antibody chains at nearly the same amount of expression to be met.

The attenuated IRES sequence was used for the expression of our selection markers.

The DNA manipulations were carried out in accordance with standard methods. Using PCR technology and the Advantage-HF PCR Kit (CLONTECH Laboratories Inc., Palo Alto, USA), the heavy and light chains of the HE2 antibody were amplified. Firstly, primer sequences were used to introduce the desired restriction sites necessary for the insertion of the gene in the expression vectors, and secondly KOZAK sequences were inserted upstream of the open reading frames.

The autologous signal sequences were used to direct the naked polypeptide chains into the secretory circulation. The primers were purchased from MWG-Biotech AG, Germany. A two-step cloning technology was developed: The Kappa chain containing its autologous signal sequence was amplified as a Xho I, Mlu I fragment and ligated into the expression vector using “Rapid Ligation Kits” (Roche, Germany) according to the manufacturer's instructions. A chemically competent E. coli bacterium strain DH5alpha (Gibco-BRL) was transfected with the construct and amplified using an ampicillin selection marker. In a second step, the reconstructed IRES sequence and the gamma-chain, which also contained the autologous signal sequence, were amplified as Mlu I, Nco I and Nco I, Sal I fragments and, in a single-step ligation reaction, were ligated into the modified expression vector already containing the HE2 Kappa chain. This construct was amplified using the E. coli bacterium strain DH5alpha (Gibco-BRL). 25 constructs originating from different PCR samples were digested with the restriction endonucleases EcoRI and BamHI. Those constructs which showed the correct restriction pattern were bidirectionally sequenced. The selection cassette described below was inserted in this expression construct. The selection marker DHFR was amplified as a PCR Xba I/Not I fragment from the pSV2-dhfr plasmid (ATCC #37146). PCR primers introduced these restriction sites. The attenuated IRES at. sequence was amplified by PCR from pSV-IRES (Clontech #6028-1) as a Sal I/Xba I fragment. In a single-step ligation reaction, IRES at. and DHFR were ligated into the already described expression construct after digestion with the respective restriction endonucleases and a further dephosphorylation step.

After a transfection of the E. coli bacterium strain DH5alpha (Gibco-BRL), positive transformants were screened by PCR. The constructs were bidirectionally sequenced and used for further transfections of eukaryotic cells.

### Example 6

### Transfection

The characterized eukaryotic strain, CHO (ATCC-CRL9096), was transfected with the above-described expression vector. To this end, the DHFR selection marker was used in order to establish stable cell lines expressing rHE2. In a 6-well cell culture plate, the cell line at cell densities of 10⁵ cells in 2 ml complete Iscove's modified Dulbecco's Medium was adjusted with 4 mM L-glutamine to a content of 1.5 g/L sodium bicarbonate and sowed upon supplementation with 0.1 mM hypoxanthin and 0.016 mM thymidine, 90%; fetal bovine serum, 10% (Gibco-BRL). The cells were allowed to grow until a cell density of 50%. In the absence of serum, the cells were then transfected with 2 μg DNA according to the manufacturer's instructions, using Lipofectin®reagent (Gibco-BRL). The transfection was stopped by the addition of complete medium after 6 or 24 hours.

### Example 7

### Selection of Positive Transformants and Cultivation

Complete medium was replaced with selection medium 24 or 48 hours after transfection. The FCS in the complete medium was replaced with dialyzed FCS (Gibco-BRL, origin: South America). Positive transformants appeared as rapidly growing multi-cellular conglomerates 10 days after the selection. The concentration of rHE2 was analyzed in the supernatants by specific sandwich ELISAS recognizing both the variable and the constant domains of the antibody. Those cells which showed high productivity were divided 1:10 and placed in 75 cm² cell culture flasks for storage in liquid nitrogen. In parallel, these producers were subjected to rising selection pressures by adding methothrexate to the culture medium, and the cells were sowed in a 6-well cell culture plate. The method was repeated approximately two weeks later, when the cells had reached a stable growth kinetics. Departing from a concentration of 0.005 μM, the MTX concentration was doubled at each selection circle until a final concentration of 1,280 μM MTX and, at the same time, subcultivation was effected in 96-well cell culture plates. The supernatants were assayed once a week by a specific sandwich ELISA which recognizes both the variable and the constant domains of the antibody. Stable cultures exhibiting the highest productivities were transferred into 75-cm² cell culture flasks and stepwise transferred in 860-cm² rolling cell culture flasks in nonselective medium. The supernatants were harvested, centrifuged, analyzed and subjected to further purification.

### Example 8

### Analysis of Expression Products

The supernatants were assayed by a specific sandwich ELISA which recognizes both the variable and the constant domains of the antibody. The polyclonal, anti-idiotypical antibody IGN111 was coated with a concentration of 10 μg/ml on Maxisorp® (NUNC) adsorption plates. The antibody was formed in goats immunized with HE2 fragments and extracted by a two-step chromatographic method by affinity. Antibodies against the constant regions of mouse were adsorbed on a polyclonal mouse IgG column in a first step, anti-idiotypical antibodies were captured by affinity on a HE2 agarose column in a second step. The final product, the polyclonal IGN111 antibody preparation, consequently recognizes the variable domain of the HE2 antibody. The remaining active groups were blocked by incubation with 1% milk powder, and the supernatants were applied. The expressed antibodies were detected through their constant regions via rabbit anti-mouse IgG2a-HRP conjugates (Biozym). Quantification was effected by comparison with a HE2 standard hybridoma antibody also packed on the column and characterized.

The size determination of the expressed proteins was effected by means of SDS polyacrylamide gel electrophoresis using 4-14% acrylamide gradient gels in a Novex® (Gibco-BRL) electrophoresis chamber. The proteins were silver-stained.

In order to immunologically detect the expressed antibodies, Western blots were carried out on nitrocellulose membranes (0.2 μm). The proteins separated by the SDS polyacrylamide gels were electrotransferred using a Novex® (Gibco-BRL) blotting chamber. The membranes were washed twice before the addition of the block solution (TBS+3% milk powder BBL) and the antibody solution (10 μg/ml polycolonal goat IGN-111 antibody, mouse monoclonal anti-mouse IgG antibody (Zymed) or rabbit anti-mouse IgG gamma-chain (Zymed) in TBS+1% milk powder). At the end, the development was performed using rabbit anti-goat HRP, rabbit anti-mouse IgG-HRP or mouse anti-rabbit IgG-HRP conjugated antibody (BIO-RAD), diluted to 1:1000 in TBS+1% milk powder, and an HRP color development reagent (BIO-RAD) was added according to the manufacturer's instructions.

Isoelectric focusing gels were used to compare the purified expression products with the characterized murine HE2 standard hybridoma antibody. The samples were loaded on IEF gels, pH 3-7 (Invitrogen), and the separation was carried out according to the manufacturer's instructions.

The proteins were visualized by silver-staining or immunological methods by means of Western blots. To this end, the proteins were loaded in a Tris-buffered SDS/urea/iodoactamide buffer and transferred onto nitrocellulose membranes. This was effected according to the same method as described for Western blots. The detection was effected by the aid of polyclonal goat IGN111 anti-idiotypical antibodies.

The interaction of the expression product with the target antigen, EpCAM, was analyzed in that the purified supernatants were incubated with nitrocellulose membranes to which rEpCAM had been electrotransferred. Staining of the interacting antibodies was carried out in a manner analogous to Western blots, using an anti-mouse IgG2a-HRP-conjugated antibody (Zymed).

### Example 9

### Affinity Purification

A Pharmacia (Amersham Pharmacia Biotech) AKTA system was used. 1000 ml of clear culture supernatant containing the antibody were concentrated with a Pro-Varion 30 kDa cut-off (Millipore) concentrator, then diluted with PBS and packed on a 20 ml IGN111 Sepharose affinity gel XK26/20 column (Amersham Pharmacia Biotech). Contaminating proteins were removed by a washing step with PBS+200 mM NaCl. The bound antibodies were eluted with 100 mM glycine, pH 2.9, and immediately neutralized with 0.5M NaHCO₃. The effluent was observed online at λ 215 and λ 280 nm and subjected to a subsequent HPLC analysis with a ZORBAX G-250 (Agilent Technologies) column.

2,000 ml of harvested supernatants from the roller bottle cultures were centrifuged, concentrated, diluted in PBS and purified to homogeneity by affinity chromatography using an IGN111 Sepharose column. After elution, neutralization and dialysis against PBS, the final product was measured by SEC-HPLC. A hybridoma-derived murine standard of the same immunoglobulin was compared with rHE2 and eluted, both simultaneously as sharp single peaks correlating with the expected retention time of IgG. A purity of >92% was obtained by this purification performed on a laboratory scale.

A further characterization of the expression product was effected by reducing and non-reducing silver-stained SDS-PAGES and Western blots. The expression products were detected by the specific anti-idiotypical antibodies, goat anti-HE2, IGN111, and visualized by an anti-goat HRP-conjugated antibody. Nonreduced samples showed bands in the expected range of an intact IgG molecule, in the region of 160 kDa. This result correlates exactly with the murine standard HE2 hybridoma antibody. With the reduced samples, bands in the range of 25 to 50 kD, also interacting with the anti-idiotypical goat anti-HE2 antibody IGN111, are visible. These bands correspond to the light and heavy chains of IgG.

The interaction with the target antigen of HE2, EpCAM, was analyzed in that nitrocellulose membranes onto which rEpCAM had been electroblotted were incubated with purified expression products. A further subtype-specific detection with interacting antibodies was carried out. The murine HE-2 standard hybridoma antibody recognizes monomeric rEpCAM of 25 kDa and also a series of rEpCAM aggregates corresponding to dimeric, trimeric and polymeric forms. Exactly the same band distribution was obtained with all purified expression products.

The purified expression products and the murine HE-2 standard hybridoma antibody were further investigated. All antibodies showed inhomogenous polyband isoelectric focusing patterns identical in terms of pH, yet different in terms of quantitative distribution. They consist of three main protein isoforms and two subforms, which are distributed over a pH range of from 8.2 to 7.2. CHO-derived isoforms were displaced to higher pH values, the murine HE2 standard showed identical isoforms, but the quantitative distribution tended to acidic forms.

The recombinant mouse IgG2a antibody HE2 could be expressed in CHO cells. The stable genomic integration occurred 14 days after transfection. The expression construct enables a rapid and easy transfection with a single plasmid. By using the selection system based on a host system that lacks an essential metabolic enzyme, the number of copies of a plasmid with the corresponding gene and a strong antagonist of this enzyme can be increased by a continuously rising selection pressure. The use of an attenuated IRES sequence in the expression cassette of this selectable marker allows the use of tiny amounts of the antagonist MTX for the selection strategy. Moderate expression was reached with amounts of 10 μg/24 hrs·ml, which could be left in the production cultures for at least 5 weeks. Purified expression products do not differ from the murine HE2 standard in size and specific immunologic assays. Nevertheless, differences may occur in the post-translatory modifications. Recombinant antibodies, therefore, show host- or media-specific isoelectric focusing patterns. The biological equivalence of the expression product was, therefore, analyzed in further immunization studies.

### Example 10

### Immunization Studies

A. 17-1A Reference Group

The murine IgG2a antibody 17-1A (17-1A) produced by hybridoma technology was purchased from Glaxo as a 10 mg/ml PBS solution under the name of Panorex®. This antibody was used as a murine standard HE2 hybridoma antibody.

B. rHE2

Recombinant HE2 was Produced as Described Above.

C. Deglycosylated 17-1A

20 mg 17-1A were deglycosylated under non-denaturizing conditions using PNGase-F (New England Biolabs, #P0704S). The completeness of the deglycosylation was controlled by Western blot analysis and by incubation with ConA peroxidase (Sabio #180705L1205-2). Buffer exchange and purification were effected by SEC Superdex 200 chromatography with 1 mM NaH₂PO₄, 0.86% NaCl, pH 6.0.

D. UPC10

UPC10, an IgG2a antibody of completely different specificity was purchased from Sigma (#M9144-1).

Vaccine Formulation

The vaccine solutions were formulated in 1 Al(OH)₃ suspensions containing 50 μg antibody/dose. The antibody solutions were assayed for their endotoxin content by the LAL endpoint method. 10 and 100 μl supernatant of the solution were tested according to the manufacturer's instructions and compared with an endotoxin standard of 0.15 to 1.2 EU/ml. Antibody solutions were dialyzed against the formulation buffer 1 mM NaH₂PO₄, 0.89% NaCl, pH 6.0 by means of a Slide-A-Lyzer Dialysis Cassette 3500 MWCO, 3-15 ml (PIERCE, #0066110). The concentration and integrity of the protein were assayed by SEC-HPLC (Zorbax-GF250, Agilent).

Immunizing Strategy

Four Rhesus monkeys (macacca mulatta) per group with body weights ranging between 4 and 6 kg were inoculated with 500 μl/animal s.c. on days 1, 15, 29 and 57 without pretreatment. Serum samples were collected on days 11, 5 and 1 (preserum), day 14, day 29, day 57 and day 71.

Blood samples for the serum preparation were collected in tubes with coagulation activator and centrifuged at 1500 g for 30 minutes (according to the instructions for use). The serum samples were transferred into tubes and stored at −80° C.

17-1A-ELISA

Presera and immune sera were analyzed by means of an ELISA test system including an immunization agent for the testing of the induced immune response. 17-1A was used as a coating antibody in a concentration of 10 μg/ml on Maxisorp®(NUNC) sorption plates, diluted with coating buffer (PAA, Lot: T05121-436). The remaining active groups were blocked by incubation with 3% FCS (Gibco-BRL, heat-inactivated, #06Q6116K) in BPS, before the sera were applied in 6×1:10 dilutions in PBS supplemented with 2% FCS. The induced antibodies were detected though their constant regions by the aid of a rabbit anti-human IgG, A, M-HRP conjugate (Zymed). Staining was effected according to usual methods. The extinction at 492 nm was measured with 620 nm as reference. Quantification was performed by a comparison with standard immune sera containing standardized antibody amounts comparable to an antibody titer of 9000.

Affinity Purification

An ÄKTA system (Amersham Pharmacia Biotech) was used. 1 ml serum was diluted 1:10 with PBS running buffer supplemented with 200 mM NaCl, and packed on a 1.0 ml 17-1A or rEpCAM Sepharose affinity gel XK10/2 column (Amersham Pharmacia Biotech) in order to specifically purify the induced overall immune reaction or the target antigen.

The contaminating proteins were removed by a washing step with PBS+200 mM NaCl. The bound antibodies were eluted with 100 mM glycine, pH 2.9 and immediately neutralized with 0.5M NaHCO₃. The effluate was measured online at λ 215 and λ 280. After this, the eluted fractions were subjected to HPLC analysis to determine the IgG/IgM ratio, purity and concentration.

Results

Taking into consideration all vaccinations, no side-effects were observed. In this immunization study, vaccinations with different IgG2a formulations in all cases led to strong antigen-specific immunization reactions of the IgG type. With the exception of the deglycosylated 17.1A formulation, which led to a weaker immune response, the immunogenity of all other formulations was nearly the same. Immune titers increased from values below the detection limit to 300 μg/ml serum, which corresponds to an induced IgG rate of almost 1%. The immunogenities of all glycosylated IgG2a antibodies used were almost in the same range irrespective of their specificities.

Likewise, irrespective of the immunization group, all IgG2a-vaccinated animals developed immune responses of the IgG type recognizing EpCAM with 30-40% of the immunization-specific antigen titer. The vaccination with IgG2a antibodies, therefore, led to a cross reactivity of the immune serum with EpCAM. The deglycosylation of the immunizing antigen significantly lowered the two IgG levels induced, both that directed against the immunizing antigen and that directed against EpCAM.

Deglycosylation clearly changed the immunogenic properties of the antibody. Immunoglobulin titers both against the immunizing antigen and against the target antigen were reduced.

A comparison between the original immunization antigen 17-1A derived from hybridoma and the recombinantly expressed rHE2 from CHO cells showed no immunological differences. Both formulations exhibited identical kinetics in the formation of specific immune responses against the immunizing antigen and the target antigen. The IgG and IgM titers formed were similar.

### Example 11

### Expression of a Hybrid Immunogenic Antibody

The recombinant IgG2a Le-Y antibody is an IgG2a hybrid antibody for primate vaccination. It combines the anti-idiotypical Lewis-Y (Le-Y) imitating (mimicking) hypervariable region and the highly immunogenic mouse-IgG2a constant regions.

The recombinant IgG2a Le-Y antibody immunotherapy increases the immunogenity of the original antibody IGN301 produced by a hybridoma cell. It induces a strong immune response against Le-Y and/or EpCAM overexpressed or presented by epithelial tumor cells. This immune response leads to the lysis of tumor cells by complementary activation or to the prevention of cell-mediated metastasization.

Molecular biological constructs of the recombinant IgG2a Le-Y antibody were inserted in the polycistronic vector.

The recombinant IgG2a Le-Y antibody was transiently expressed in HEK293 cells, after this calcium-phosphate co-precipitation took place in a micro spin system in the presence of FCS. After purification by the aid of an anti-Le-Y affinity column and qualification of the expression product, the recombinant IgG2a Le-Y antibody was formulated on Al(OH)₃ and used as a vaccine in Rhesus monkey immunization studies at four 500-μg doses.

A high immunogenity as compared to that of the original IGN301 vaccine was to be observed. The induced immune response of the IgG type was analyzed by ELISA and showed immunization antigen, Le-Y and EpCAM, specificities.

1. An immunogenic antibody which comprises at least two different epitopes of a tumor-associated antigen, one epitope being derived from the group of peptides or proteins and one epitope being derived from the group of carbohydrates.

2. The antibody according to claim 1, characterized in that it comprises at least one epitope of an antigen selected from the group consisting of peptides or proteins, carbohydrates, and glycolipids.

3. The antibody according to any one of claims 1 or 2, characterized in that it comprises at least two epitopes of EpCAM.

4. The antibody according to claim 1, characterized in that it is conjugated with a: peptide, glycopeptide, carbohydrate, lipid or nucleic acid.

5. The antibody according to claim 4, characterized in that said peptide, glycopeptide, carbohydrate, lipid or nucleic acid represents an epitope of a tumor-associated antigen.

6. The antibody according to claim 1, characterized in that it comprises at least one epitope of EpCAM and at least one epitope of Lewis Y.

7. The antibody according to claim 1, characterized in that it comprises at least one epitope of EpCAM and at least one epitope of sialylTn.

8. The antibody according to claim 1, characterized in that it is a human, humanized, chimeric or murine antibody.

9. The antibody according to claim 1, characterized in that it is a recombinant antibody.

10. The antibody according to claim 1, characterized in that it is an antibody derivative selected from the group consisting of antibody fragments, conjugates or homologs.

11. The antibody according to any one of claim 1, characterized in that it comprises a specificity for a tumor-associated antigen.

12. The antibody according to claim 11, characterized in that it comprises a specificity for an antigen selected from the group consisting of EpCAM, NCAM, CEA and Lewis Y.

13. The antibody according to claim 11, characterized in that it comprises a specificity for EpCAM and at least one epitope of EpCAM, particularly at least two epitopes of EpCAM.

14. The antibody according to claim 1, characterized in that it comprises a specificity for an antibody.

15. The antibody according to claim 13, characterized in that it is an anti-idiotypical antibody.

16. The antibody according to claim 15, characterized in that it recognizes the idiotype of an antibody against a tumor-associated antigen.

17. The antibody according to claim 16, characterized in that said antigen is selected from the group consisting of peptides or proteins, carbohydrates, glycolipids.

18. A pharmaceutical preparation comprising an immunogenic antibody according to claim 1.

19. A diagnostic agent comprising an immunogenic antibody according to claim 1.

20. A vaccine formulation comprising an immunogenic antibody according to claim 1.

21. A vaccine formulation according to claim 20, characterized in that said antibody is contained in an immunogenic amount of 0.01 μg to 10 mg.

22. A vaccine formulation according to any one of claims 20 or 21, characterized in that at least one vaccine adjuvant is contained.

23. A method for producing an immunogenic antibody according to claim 1, by
a) providing an antibody including the idiotype of a tumor-associated antigen; and
b) coupling at least one epitope of a tumor-associated antigen or its mimicry to said antibody.

24. A method for producing an immunogenic antibody according to claim 1, by
a) providing an antibody; and
b) coupling at least two epitopes of a tumor-associated antigen or its mimicry to said antibody.

25. A method for producing an immunogenic antibody according to claim 1, by
a) providing a nucleic acid encoding an antibody including the idiotype of a tumor-associated antigen; and
b) recombining said nucleic acid with a nucleic acid encoding an epitope of a tumor-associated antigen or its mimicry.

26. A method for producing an immunogenic antibody according to claim 1, by
a) providing a nucleic acid encoding an antibody; and
b) recombining said nucleic acid with one or several nucleic acid(s) encoding at least two epitopes of a tumor-associated antigen or its mimicry.

27. A method according to any one of claims 23 to 26, characterized in that said antibody is ab1 and/or ab2.

28. A method for producing an immunogenic antibody according to claim 1, characterized in that an epitope of a tumor-associated antigen or its mimicry is conjugated to said antibody as a carrier.

29. A method according to claim 28, characterized in that said antigen is selected from the group consisting of peptides or proteins, carbohydrates, and glycolipids.

30. A method according to claim 28 or 29, characterized in that a nucleic acid encoding an epitope of a peptide or protein antigen is conjugated to said antibody.

31. A method according to any one of claims 28 to 29, characterized in that said antibody comprises at least one further epitope of a tumor-associated antigen.

32. The immunogenic antibody according to claim 2 or 17, wherein the peptide or protein is selected from the group consisting of EpCAM, NCAM, CEA and T-cell peptides, the carbohydrate is selected from the group consisting of Lewis Y, sialylTn, GloboH, and the glycolipids are selected from the group consisting of GD2, GD3 and GM2.

33. The method according to claim 29, wherein the peptide or protein is selected from the group consisting of EpCAM, NCAM, CEA and T-cell peptides, the carbohydrate is selected from the group consisting of Lewis Y, sialylTn, GloboH, and the glycolipids are selected from the group consisting of GD2, GD3 and GM2.
